(19) United States
(12) Patent Application Publication
(10) Pub. No.: US 2006/0019399 A1
(43) Pub. Date: Jan. 26, 2006

(54) Devices and methods for correlated analysis of multiple protein or peptide samples

(76) Inventors: Brian M. Balgley, Annandale, VA (US); Jonathan W. Cooper, Walkersville, MD (US); Cheng S. Lee, Ellicott City, MD (US); Donald L. DeVoe, Bethesda, MD (US)

Correspondence Address: Donald L. DeVoe, 5619 Sonoma Road, Bethesda, MD, 20817, US

(21) Appl. No.: 11/171,427

(22) Filed: Jul. 1, 2005

Related U.S. Application Data
(60) Provisional application No. 60/584,855, filed on Jul. 2, 2004.

(51) Int. Cl. G01N 33/00

(52) U.S. Cl. 436/86

(57) ABSTRACT

Disclosed is a system for performing multiple analyses of protein and/or peptide samples and correlating the results of the analyses. The system comprises a sample inlet, a splitter means, at least two sample delivery capillaries, at least two sample deposition tools, and at least two sample collectors, wherein said splitter means is in fluid communication with the sample inlet and the sample delivery capillaries, and wherein liquid flow entering the splitter means is split into a number of sub-flows equal to the number of sample delivery capillaries. In one preferred embodiment, at least one microenzyme reactor is interfaced to a first sample delivery capillary in order to digest a protein sample within the capillary, while a second sample delivery capillary does not contain a microenzyme reactor, thereby enabling correlated analysis of the same protein sample in digested and undigested form. Methods for performing two or more analyses of protein and/or peptide samples and correlating the results of the analyses are also disclosed.

### CROSS-REFERENCE TO RELATED APPLICATIONS

This application claims priority from U.S. Provisional Patent Application Ser. No. 60/584,855, filed Jul. 2, 2004, which is incorporated herein by reference in its entirety.

This invention was made with government support under Grant No. 0319722 from the National Science Foundation, and Grants No. R44 RR019108 and R43 CA 103086 from the National Institutes of Health. The government may have certain rights to this invention.

### BACKGROUND

1. Field of Invention

The invention relates to devices and methods for providing an interface between capillary separations and mass spectrometry for the purpose of performing correlated analyses between separated protein and/or peptide samples following differential treatment.

2. Background of the Invention

Proteomic analysis by mass spectrometry is typically accomplished by one of two methods, commonly referred to as “bottom-up” and “top-down” proteomics. Broadly speaking, bottom-up analysis indicates the protein sample has been enzymatically digested into constituent protein fragments or peptides prior to introduction into a mass spectrometer (Yates et al., 1995). In contrast, top-down analysis indicates the protein sample is introduced intact into a mass spectrometer (Ge et al., 2002).

One type of bottom-up analysis allows a mixture of peptides to be assigned to their originating protein by a database search algorithm termed peptide mass fingerprinting (Pappin et al., 1993). Essentially, a protein is digested by an enzyme, typically trypsin, and the resulting peptide mixture is introduced into a mass spectrometer. The masses of the ionized peptides are recorded and an attempt is made to match those masses with the masses predicted from an in silico enzymatic digestion of protein sequences in a database. This approach is useful for a purified protein, often isolated from a gel-based separation. Using this approach, existing search algorithms typically fail to identify mixtures of greater than a few proteins. Because a sample containing multiple proteins produces far more peptides than a single protein, a different approach is required. The bottom-up approach for analyzing a more complex mixture of proteins involves enzymatically digesting the proteins and then separating the resulting peptides, e.g. with liquid chromatography, prior to introduction into a mass spectrometer. The peptides, once in the mass spectrometer, are fragmented and the fragment ion masses are measured. These fragmentation patterns can be used to deduce a peptide sequence using one of several available search algorithms. The search algorithms attempt to match the experimental fragmentation patterns with patterns predicted from peptide sequences in a protein sequence database. A drawback of the bottom-up approach is that the data output is a list of peptides with varying probabilities of identification certainty. Protein information must be inferred from the presence of constituent peptides. Typically, one to three peptides are used to infer the presence of a protein, meaning sequence coverage of that protein is very low compared to peptide mass fingerprinting results. It is unlikely that peptides will be identified to confirm cases where the protein is post-translationally modified or is an expression variant. In both types of bottom-up analyses the proteins may be enzymatically digested either prior to introduction to an inlet in-line with the mass spectrometer or after introduction to an inlet in-line with the mass spectrometer through the use of an enzyme reactor. These bottom-up approaches provide very limited molecular information about the intact proteins, particularly towards the detection of post-translational modifications (PTMs). PTMs include co- or post-translation covalent modifications to the protein structure and proteolytic processing of the translated protein. Such modifications may be overlooked in analyses using peptide based (bottom-up) approaches, where only a fraction of the total theoretical peptide population of a given protein may be identified.

Top-down proteomic analysis typically consists of introducing an intact protein into a mass spectrometer and fragmenting the protein. The resulting fragmentation is orders of magnitude more complex than a peptide fragmentation, necessitating the use of a mass spectrometer with very high mass accuracy and resolution capability in order to interpret the fragmentation pattern with acceptable certainty. A search algorithm for protein fragmentation compares the experimental intact protein mass and the fragments generated with those predicted from a protein sequence database. Another search algorithm compiles experimental sequence tag data and attempts to match the predicted fragment sequences to those in a protein sequence database. An advantage of a top-down analysis over a bottom-up analysis is that a protein may be identified, rather than inferred as is the case with peptides. Another advantage is that alternative forms of a protein, e.g. post-translational modifications and splice variants, may be identified. However, a disadvantage when compared to a bottom-up analysis is that many proteins can be difficult to isolate and purify into conditions suitable for mass spectrometric analysis. Another disadvantage is the requirement for a very high mass accuracy and resolution mass spectrometer, typically a Fourier-transform ion-cyclotron resonance MS, which are currently expensive to purchase and operate. Furthermore, proteins are less ionizable than peptides for mass spectrometry detection, thus requiring significantly higher amount of protein samples to perform the analysis. This large sample requirement may be impractical for studies of protein profiles within small cell populations or limited tissue samples and adversely impacts the ability to perform comprehensive proteome analysis, particularly toward the identification of low abundance proteins.

Thus a need exists for a method which can generate intact protein mass information for a subset of proteins within a complex sample, and correlate this information with peptide mass information resulting from the same subset of proteins. Ideally, this functionality should be provided in a single automated instrument which allows a series of protein samples to be sequentially analyzed. By combining such correlated top-down and bottom-up analysis data, improved protein identification can be realized over separate top-down or bottom-up analysis. Furthermore, the ability to combine correlated top-down and bottom-up analysis data can provide improved protein identification over combined top-down and bottom-up analysis of a complex sample where no correlation is provided between top-down and bottom-up data from multiple subsets of the overall sample.

An additional need relates to the analysis of PTMs. It should be emphasized that the biological processes involved in cell signaling, transcription regulation, responses to stresses, etc. are made of complex linkages that determine system properties. Protein PTMs (e.g. phosphorylation or glycocylation) can particularly modulate function and are essential to the understanding of regulatory mechanisms. The current peptide-based (bottom-up) approaches provide very limited molecular information about the intact proteins, particularly towards the detection of PTMs. On the other hand, protein-based (top-down) processes are limited by their large sample requirements and poor capability towards the analysis of low abundance proteins and associated PTMs. Thus there is a need for direct correlation of peptides and their sequences with the corresponding proteins and measured protein masses, in order to improve the identification of PTMs.

One approach to mapping PTMs is through the differential display of proteins which have on one hand been proteolytically modified to elucidate a particular PTM, and on the other hand not modified, thereby allowing the identification and quantification of the PTM within the unmodified sample. This approach is often realized using differential 2-D gel analysis, for example by dividing an initial protein sample into two aliquots, with one aliquot run via 2-D PAGE following dephosphorylation using a phosphotase, the other run via 2-D PAGE without further treatment, and the resulting protein patterns compared to determine shifts in visualized protein spots which are representative of changes in phosphorylation of the specific proteins between the two aliquots (Yamagata et al., 2002). Final identification of differentially-expressed proteins may be performed by excising desired protein spots from the gel, followed by MS analysis. However, many important regulatory proteins, which are expressed at extremely low levels, are precluded in the combined 2D-PAGE-MS technique unless extensive fractionation of large quantities of protein together with the processing of a large number of narrow-range gels is performed. The 2-D PAGE-MS approach also remains lacking in proteome coverage (for proteins having extreme isoelectric points or molecular masses as well as for membrane proteins), dynamic range, sensitivity, and throughput. Furthermore, while 2-D PAGE-MS may be performed using either a top-down or bottom-up approach, the ability to automatically correlate data from both approaches is currently lacking. Thus, there is a need for a method which can correlate data from analyses of proteins which have been enzymatically treated, with analyses of the same proteins which have not been treated, in order to provide improved analysis of PTMs without necessarily relying on 2-D gels for front-end separation.

The present invention fulfills these and other needs.

### SUMMARY OF THE INVENTION

One advantage of the invention is that it enables the combination of top-down and bottom-up biomolecular analysis in a single automated platform. By splitting the analyte before the reactor, both intact separated proteins and peptides from the same digested proteins may be separately analyzed by MS, with the resulting peptide data correlated to the protein data for improved protein identification.

An aspect of the invention is the correlation of data resulting from each MS analysis. For example, a stream of proteins entering the sample inlet and split into two portions by the splitter means may have one portion analyzed for intact protein masses, and the other portion digested in the microenzyme reactor and the resulting digest analyzed for peptide masses, with the resulting peptide mass measurements correlated to the intact protein mass measurements. Similarly, a stream of proteins entering the sample inlet and split into two portions by the splitter means may have one portion analyzed for intact and unmodified protein masses, and the other portion enzymatically modified in the microenzyme reactor using a phosphotase with the resulting treated proteins analyzed for intact but modified protein masses, and with data from each correlated pair of modified and unmodified mass analyses compared to determine the relative phosphorylation state of various proteins within the initial mixture.

Another aspect of the invention is the ability to use multiple enzymes within the microenzyme reactor. For example, a combination of two or more enzymes may be desirable to ensure more complete digestion of proteins passing through the reactor. Similarly, a combination of a phosphotase and a glycanase within a single microenzyme reactor may be desirable for the simultaneous analysis of phosphoproteins and glycoproteins in the same initial sample. Alternately, multiple microenzyme reactors each containing a different bound enzyme may be placed in series along a single sample delivery capillary to achieve the desired results.

In another aspect, the apparatus provides a measurement system for monitoring the positions of sample plugs within the system, enabling the time required for different portions of an initial sample plug entering the splitter means to be delivered to one or more MS interfaces along different flow paths to be determined.

These and other features and advantages of the invention will be more fully appreciated from the detailed description of the preferred embodiments and the drawings attached hereto. It is also to be understood that both the foregoing general description and the following detailed description are exemplary and not restrictive of the scope of the invention.

### BRIEF DESCRIPTION OF THE DRAWINGS

FIG. 1 is a schematic diagram of a preferred embodiment of the instrument using a single in-line microenzyme reactor.

FIG. 2 is a schematic diagram of a preferred embodiment of the instrument using multiple in-line microenzyme reactors, with separation columns provided for separation of components within each sub-flow.

### DETAILED DESCRIPTION OF THE INVENTION

I. Apparatus

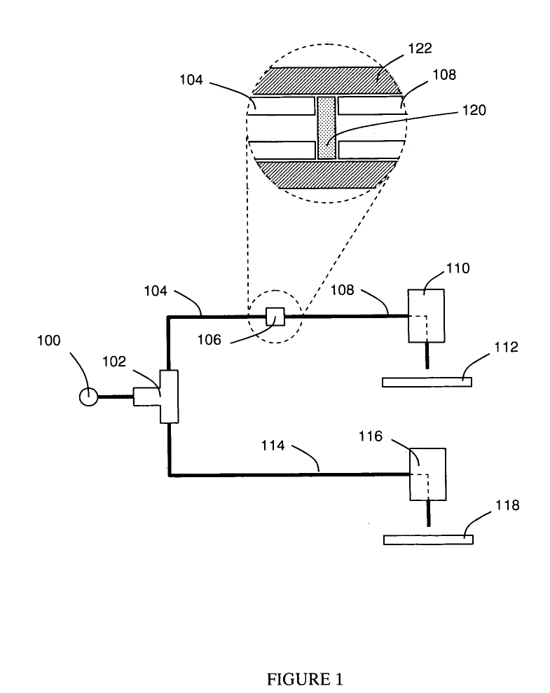

A preferred embodiment of the apparatus is depicted in FIG. 1. The apparatus comprises a sample inlet 100 which is connected to a splitter means 102. It will be appreciated that the sample inlet may provide sample from the outlet of a separation capillary, a microtiter plate, or other sample loading means. The splitter means comprises an inlet end, a first outlet end, and a second outlet end, and serves to distribute fluid entering the inlet end into sub-flows exiting each of the outlet ends. The inlet end of the splitter means is connected to the sample inlet. The first outlet end of the splitter means is connected to the first end of a first sample delivery capillary 104. A first microenzyme reactor 106 is positioned in-line with the first sample delivery capillary 104, such that the reactor intersects the flow of sample through the capillary. Referring to the inset in FIG. 1, the microenzyme reactor consists an enzyme-impregnated membrane 120 which intersects the first sample delivery capillary 104, such that the capillary is split into two parts with the membrane tightly positioned between the parts, and a sheath 122 which serves to hold the membrane/capillary assembly together. A portion of the first sample delivery capillary is connected to a first sample deposition tool 110, which is capable of positioning the outlet end of the first sample delivery capillary at different locations over a first sample collector 112. The second outlet end of the splitter means is connected to a second sample delivery capillary 114. A portion of the second sample delivery capillary is connected to a second sample deposition tool 116, which is capable of positioning the outlet end of the second sample delivery capillary at different locations over a second sample collector 118. A pressure means is also provided to mobilize sample from the sample inlet through the splitter means and sample delivery capillaries.

The capillaries may be fabricated from a number of different materials, including silica, plastics, or metals. Capillaries with inner diameters on the order of 10 μm to 100 μm may be desirable for applications involving small sample volumes, but larger or smaller inner diameters may also be used depending on the application. Furthermore, the use of the term “capillary” does not limit the invention to traditional capillary tubing. For example, the capillaries may consist of microchannels embedded in a planar microfluidic substrate fabricated from glass, silicon, plastic, or other material as commonly employed in microchannel manufacturing. Similarly, the capillary channels which carry fluid need not be circular in cross-section. For example, the channels may be ellipsoidal, rectangular, or trapezoidal in cross-section, depending on the method used for fabrication.

The sample collectors 112 and 118 may be one of several possible types, including but not limited to MALDI target plates, microtiter plates, or storage vial arrays. In each case, the method for eluting sample onto the sample collector may differ. For example, pressure mobilization of analyte mixed with matrix solution may be desirable for MALDI target plate deposition, while deposition of unmixed analyte using pressure mobilization or electrospray deposition may be desirable in other instances. Furthermore, the two sample collectors may share the same substrate, for example two portions of a single MALDI target plate may serve as the sample collectors. Similarly, the function of the two sample deposition tools may be achieved by using a single sample deposition tool, wherein both sample delivery capillaries are positioned over the sample collectors in a multiplexed fashion using a shared positioning system. Alternately, one or both of the sample deposition tools and sample collectors may be replaced by a direct interface to mass spectrometry, such as electrospray-ionization mass spectrometry (ESI-MS).

It will be appreciated that it may be desirable for the splitter means to possess more than two outlet ends, allowing analyte entering the inlet end of the splitter means to be routed to more than two mass spectrometers, with different modifications to each portion of analyte performed prior to mass spectrometry. Regardless of the number of outlet ends of the splitter means, the splitter means may be configured to achieve desired ratios for volumetric flow and flow rate through each of the outlet ends.

According to another embodiment, the first microenzyme reactor 106 is omitted, with differential analysis performed by differentially treating the samples deposited on the first 112 and second 118 sample collectors. For example, the first sample collector may consist of a MALDI target, with on-target proteolytic digestion performed to enable analysis of peptides and protein fragments, and the second mass spectrometry interface may consist of a MALDI target with no proteolytic digestion performed to enable analysis of intact proteins.

According to another embodiment a second microenzyme reactor is positioned in-line with the second sample delivery capillary 114. The second microenzyme reactor may contain a proteolytic enzyme different from the enzyme in the first microenzyme reactor, thereby enabling differential mass spectrometry analysis to be performed on eluent from each of the mass spectrometry interfaces.

According to another embodiment, detection of biomolecules is performed between the splitter means and first microenzyme reactor, between the first microenzyme reactor and first mass spectrometry interface, between the splitter means and second mass spectrometry interface, or any combination of these three, thereby enabling precise measurement of the position(s) of analyte within any flow path within the apparatus. The detector used may be a UV detector, a laser induced fluorescence detector, a conductometric detection, or any combination of these or other detection methods.

According to another embodiment, a flow detector is positioned between the splitter means and first microenzyme reactor, between the first microenzyme reactor and first mass spectrometry interface, between the splitter means and second mass spectrometry interface, or any combination of these three, thereby enabling precise measurement of flow conditions within any flow path within the apparatus.

According to another embodiment, the sample inlet is in fluid communication with a capillary wherein separation of proteins is performed, allowing separated proteins to be sequentially supplied to the sample inlet.

The splitter means may consist of a simple fluidic junction designed to split an incoming fluid flow into multiple outgoing flows, such as a capillary “Y” or “T” junction. Alternate splitter means may also be realized. One such alternate splitter means comprises a flow switch consisting of an inlet and one or more outlets, with splitting achieved by duty cycling between the various outlets to achieve the desired flow ratios.

The microenzyme reactor may contain one of a number of possible enzymes for modifying proteins which pass through the reactor. In a preferred embodiment, the reactor contains trypsin bound to a solid support in order to digest proteins passing through the reactor into constituent peptides. Alternately, the microenzyme reactor may contain an enzyme for cleaving proteins based on specific protein modifications, such as phosphotase for removing phosphorylated groups from phosphoproteins. Furthermore, it may be desirable to have more than one enzyme within a single microenzyme reactor to affect multiple modifications to proteins passing through the reactor.

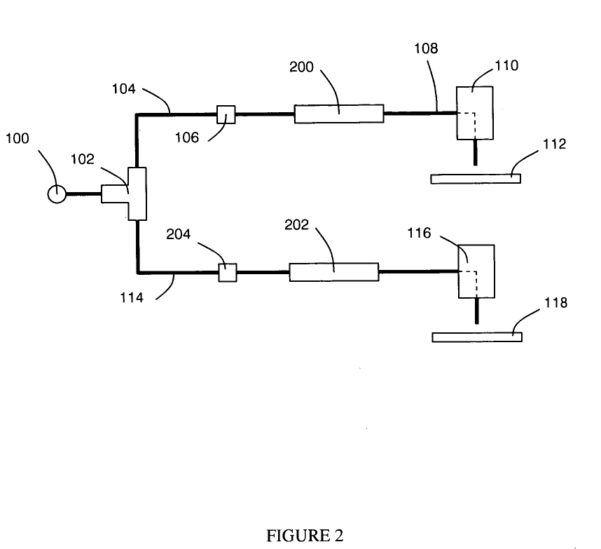

According to another embodiment, depicted in FIG. 2, a first separation column 200 may be positioned between the first microenzyme reactor 106 and the second end of the first sample delivery capillary 108. A second microenzyme reactor 204 may be positioned in-line with the second sample delivery capillary, and a second separation column may be positioned between the second microenzyme reactor and the second end of the second sample delivery capillary 114. The first and second separation columns provide a means for separating proteins, protein fragments, and/or peptides prior to deposition on the sample collectors. The separations may be based on one or more chromatographic or electrokinetic separation mechanisms, such as reverse-phase liquid chromatography, capillary gel electrophoresis, capillary zone electrophoresis, isoelectric focusing, or isotachophoresis.

II. Methods

In one aspect of the invention, a method is provided for splitting an incoming sample flow from a chromatographic or electrokinetic separation, with proteins within one of the split sub-flows enzymatically cleaved and analyzed by mass spectrometry, and proteins within the other sub-flow analyzed in their intact form by mass spectrometry. The initial sample steam is continuously split by the splitter means into two sub-flows, with one sub-flow directed through the first membrane reactor for online enzymatic cleavage of intact proteins. This first sub-flow, now consisting of protein fragments or peptides, is directed through the first mass spectrometry interface for protein fragment molecular weight determination or peptide sequence identification. The second sub-flow, consisting of intact proteins, is directed to the second mass spectrometry interface for intact protein molecular weight determination. The splitter means is configured such that the time for the first sub-flow to travel from the splitter means to the first mass spectrometry interface is substantially equal to the time for the second sub-flow to travel from the splitter means to the second mass spectrometry interface, thereby ensuring that MS analyses at each time step are correlated.

In another aspect, the invention includes a method for tracking different fractions of each sub-flow after deposition onto the sample collectors, and providing a correlation table listing which fractions on each sample collector correspond to portions of individual sub-flows which originated from the same initial sample plug introduced into the splitter means. Using this approach, the table may be consulted either manually or automatically using appropriate computer software to select paired fractions from each sample collector. Following mass spectroscopy analysis of a given set of paired fractions, the resulting MS data from each fraction may be combined to provide more comprehensive analysis than could be achieved from MS analysis of either fraction alone.

We claim:

1. An apparatus for performing two or more correlated analyses on a sequence of protein samples, said apparatus comprising a sample inlet, a splitter means, at least two sample delivery capillaries each possessing a first and second end, at least two sample deposition tools, and at least two sample collectors, wherein said splitter means comprises an inlet port and at least two outlet ports, with the inlet port in fluid communication with the sample inlet and each outlet port in fluid communication with the first end of a sample delivery capillary, and wherein liquid flow entering the inlet port is split within the splitter means into a number of sub-flows equal to the number of sample delivery capillaries.

2. The apparatus of claim 1, wherein the sample inlet is in fluid communication with the outlet end of a separation capillary, such that separated proteins enter the sample inlet sequentially.

3. The apparatus of claim 1, wherein the splitter means is a “T” or “Y” capillary connector.

4. The apparatus of claim 1, wherein the splitter means is a multi-port valve possessing an input port and two or more outlet ports, and operated under computer control such that sub-flows within the two or more outlet ports are defined by rapidly switching between the inlet port and each outlet port.

5. The apparatus of claim 1, wherein the time required for a plug of fluid to mobilize from the first end to the second end of the first sample delivery capillary is substantially equal to the time required for a plug of fluid to mobilize from the first end to the second end of the second sample delivery capillary.

6. The apparatus of claim 1, wherein the capillaries are manufactured from glass, silica, or plastic.

7. The apparatus of claim 1, wherein the capillaries are microfluidic capillaries fabricated in a planar substrate.

8. The apparatus of claim 1, wherein the capillaries possess an average inner diameter of between 10 microns and 500 microns.

9. The apparatus of claim 1, further comprising at least one microenzyme reactor located between the first and second ends of the at least one sample delivery capillaries.

10. The apparatus of claim 9, wherein the at least one microenzyme reactor comprises a porous membrane suitable for binding one or more enzymes, two capillary segments each possessing a first end and a second end, and a sleeve for receiving the porous membrane and two capillary segments, said sleeve possessing an inner diameter approximately equal to the outer diameter of the capillaries, and said porous membrane positioned between the second end of the first capillary segment and the first end of the second capillary segment.

11. The apparatus of claim 10, wherein the porous membrane comprises a polyvinylidene fluoride (PVDF) or polytetrafluoroethylene (PTFE) membrane.

12. The apparatus of claim 10, wherein the porous membrane is between 10 microns and 250 microns thick, and possesses an average pore size of between 0.1 micron and 2.0 microns.

13. The apparatus of claim 10, wherein the one or more enzymes bound to the membrane is trypsin, pepsin, chymotrypsin, elastase, or carboxypeptidase, exoglycosidase, endoglycosidase, or other proteolytic enzyme.

14. The apparatus of claim 10, wherein the one or more enzymes bound to the membrane is a kinase or phosphotase.

15. The apparatus of claim 1, wherein at least one of the sample deposition tools is a MALDI target spotting tool, and wherein at least one of the sample collectors is a MALDI target.

16. The apparatus of claim 1, wherein at least one of the sample deposition tools is a microfraction collector, and wherein at least one of the sample collectors is a microtiter plate.

17. The apparatus of claim 1, wherein at least one of the sample deposition tools is an electrospray ionization emitter.

18. The apparatus of claim 17, wherein at least one of the sample collectors is replaced by a mass spectrometer compatible with ESI-MS analysis.

19. The apparatus of claim 1, further comprising one or more biomolecule detectors located between the first and second ends of the at least two sample delivery capillaries.

20. The apparatus of claim 19, wherein the one or more biomolecule detectors are optical detectors.

21. The apparatus of claim 1, further comprising one or more flow detectors located between the first and second ends of the at least two sample delivery capillaries.

22. The apparatus of claim 1, further comprising one or more separation columns positioned between the first and second ends of the two or more sample delivery capillaries, such that biomolecules within the sample delivery capillaries may be further separated.

23. A method for performing multiple correlated analyses on a sequence of protein samples, said method comprising
a. providing an assembly which includes a sample inlet, a splitter means, at least two sample delivery capillaries each possessing a first and second end, at least two sample deposition tools, and at least two sample collectors, wherein said splitter means comprises an inlet port and at least two outlet ports, with the inlet port in fluid communication with the sample inlet and each outlet port in fluid communication with a sample delivery capillary, and wherein liquid flow entering the inlet port is split into a number of sub-flows equal to the number of sample delivery capillaries,
b. applying a series of one or more initial sample components within a sample stream to the sample inlet,
c. applying a pressure to the sample inlet, under conditions effective to cause said initial sample components to mobilize from the sample inlet, through the splitter means, and into the one or more sample delivery capillaries, thereby producing a number of discrete sub-flows equal to the number of sample delivery capillaries,
d. further applying a pressure to the sample inlet, under conditions effective to cause each sub-flow to mobilize through its corresponding sample delivery capillary.

24. The method of claim 23, further comprising detecting the time at which sample components pass one or more locations within each sample delivery capillary.

25. The method of claim 23, further comprising measuring the rate of liquid flow through each sample delivery capillary.

26. The method of claim 23, further comprising providing at least one microenzyme reactor positioned in-line with one or more sample delivery capillaries such that substantially all biomolecules mobilized through said sample delivery capillaries pass through the at least one microenzyme reactor.

27. The method of claim 23, further comprising
a. providing at least one separation column positioned in-line with one or more sample delivery capillaries and adapted to perform capillary electrophoresis,
b. applying a pressure to the sample inlet, such that a plug of sample solution substantially fills the separation column,
c. removing the applied pressure to the sample inlet,
d. applying a first voltage gradient across the length of the separation column, such that different components become separated at least partially on the basis of size,
e. re-applying a pressure to the sample inlet, such that the separated components within the separation column are replaced by another plug of sample solution, thereby allowing the separation process to be repeated.

28. The method of claim 23, further comprising
a. providing at least one separation column positioned in-line with one or more sample delivery capillaries and adapted to perform capillary isoelectric focusing,
b. applying a pressure to the sample inlet, such that a plug of sample solution substantially fills the separation column,
c. removing the applied pressure to the sample inlet,
d. applying a first voltage gradient across the length of the separation column, such that different components become separated at least partially on the basis of isoelectric point,
e. re-applying a pressure to the sample inlet, such that the separated components within the separation column are replaced by another plug of sample solution, thereby allowing the separation process to be repeated.

29. The method of claim 23, further comprising
a. providing at least one separation column positioned in-line with one or more sample delivery capillaries and adapted to perform capillary liquid chromatography,
b. applying a pressure to the sample inlet, such that a plug of sample solution substantially fills the separation column,
c. removing the applied pressure to the sample inlet,
d. applying a pressure gradient across the length of the separation column, such that different components become separated at least partially on the basis of surface interactions with a chromatographic medium within the separation column,
e. re-applying a pressure to the sample inlet, such that the separated components within the separation column are replaced by another plug of sample solution, thereby allowing the separation process to be repeated.

30. The method of claim 23, further comprising sequentially eluting fractions of the sub-flow from the second end of at least one sample delivery capillary using a sample deposition tool.

31. The method of claim 30, further comprising depositing the said one or more fractions of the sub-flow from the at least one sample delivery capillary onto a sample collector.

32. The method of claim 23, further comprising
a. providing suitable components of the assembly such that the time required for a fraction of fluid to mobilize from the first to the second end of the first sample delivery capillary is known, and the time required for a fraction of fluid to mobilize from the first to the second end of the second sample delivery capillary is known,
b. sequentially depositing fractions from each sample delivery capillary onto the at least one sample collector,
c. recording the location, start time, and stop time for each fraction deposition,
d. calculating the time at which the front end of each fraction entered the splitter means, and the time at which the back end of each fraction entered the splitter means.
e. constructing a list of deposited fractions which entered the splitter means at least in part simultaneously, such that fractions which contain portions of the same initial sample may be determined from said list.

33. The method of claim 32, wherein the time required for a fraction of fluid to mobilize from the first to the second end of the first sample delivery capillary is substantially equal to the time required for a fraction of fluid to mobilize from the first to the second end of the second sample delivery capillary.
